(19) United States
(12) Patent Application Publication
(10) Pub. No.: US 2021/0047598 A1
(43) Pub. Date: Feb. 18, 2021

(54) Centrifuge Fermenter Array

(71) Applicants: Andre Alexandre Simoes, San Marcos, CA (US); Jeffrey Brandon Guziejka, Aliso Viejo, CA (US)

(72) Inventors: Andre Alexandre Simoes, San Marcos, CA (US); Jeffrey Brandon Guziejka, Aliso Viejo, CA (US)

(73) Assignee: MicroBio Products LLC, Aliso Viejo, CA (US)

(21) Appl. No.: 16/538,539

(22) Filed: Aug. 12, 2019

(51) Int. Cl. C12M 1/32; B01L 3/00

(52) U.S. Cl. CPC C12M 23/12; B01L 3/5085; B01L 2300/0851; B01L 2400/086; B01L 2300/0893; B01L 2300/0854; B01L 2300/12; B01L 2300/0858

(57) ABSTRACT

A centrifuge microplate fermenter for culturing cells in a nutrient medium wherein the centrifuge microplate fermenter is comprised of a top end, a bottom end, a vertical cylindrical outer wall positioned between the top end and the bottom end, and an inner wall positioned inside the outer wall, the bottom end further comprised of a plurality of baffles which protrude from inner wall toward a theoretical center of the vertical cylindrical outer wall terminating in a conical tip

### RELATED APPLICATIONS

This application is a non-provisional patent application claiming the benefit of priority from U.S. Provisional Pat. App. Ser. No. 65/584,792 filed Nov. 11, 2017 the entire contents of which are incorporated by reference herein.

### FIELD OF THE INVENTION

The present invention pertains generally to centrifuge microplates. More particularly, the present invention pertains to a centrifuge fermenter array with each “well” having side and bottom baffles.

### BACKGROUND OF THE ART

The centrifuge microplate is a flat plate with multiple “wells” used as small test tubes. The centrifuge microplate has become a standard tool in analytical research and has numerous applications in different fields of biotechnology. In general, centrifuge microplates are rectangular with an array of individua wells each typically conical, rounded or square and closed at one end. Such centrifuge microplates are capable of holding 0.4 to 2.0 ml samples, often liquids, and are designed to withstand forces in excess of 10,000 times their own weight (10,000×g) during centrifugation. The centrifuge microplates are used widely in laboratories as vessels for holding radioisotope chemicals, for storing biochemical, for performing biochemical reactions, and for handling contaminant free samples.

As is often the case in biotechnology research, performing biochemical reactions requires the rapid increase in volume of biological starting materials. Rapid growth of host organisms such as E. coli, S. cerevisiea, and Pichia in small vessels is limited because of poor nutrient and gas exchange during growth. For example, recovery after Plasmid mediated transformation of frozen competent E. coli is improved when cells are placed in larger centrifuge microplates. This growth rate enhancement can increase the transformation efficiency by a factor of 25-fold for DH10B cells.

One alternative vessel for culturing cells is the fermentation flask, such as that disclosed by Ellis and Harlan in US. Pat. Pub. No. US2005/277191. The bottom of the flask has six equally spaced baffles extending inward from the rounded corners of the flask and upward toward a large diameter opening of the flask centered on the longitudinal axis. The baffles have a triangular cross-sectional shape with an included angle of about 28-40° and a height measured parallel to the longitudinal axis of about 15-25% of the usable flask volume. Although available, increasing the size of the culture vessel is not always a viable option or may not be the desired approach when starting material volumes are extremely small.

There are no examples of centrifuge microplates with baffled “wells” and it is an object of the present invention to provide the desired features described herein as well as additional advantages such as improving and expediting the living cell production growth rate and resulting plasmid yield by over 60%/unit volume.

### SUMMARY OF THE INVENTION

The present invention is directed to solving the deficiency in the art pertaining to the availability of centrifuge microplate fermenters. It is one object of the present invention to provide a centrifuge microplate fermenter with each “well” having side and bottom baffles.

It is another object of the present invention to provide a centrifuge microplate fermenter with each “well” having side and bottom baffles capable of improving and expediting the living cell production growth rate and resulting plasmid yield by over 60%/unit volume.

It is another object of the present invention to provide a centrifuge microplate fermenter with each “well” having side and bottom baffles wherein during agitation the baffles cause a greater percentage of culture medium and/or organism surface area to be exposed to oxygen. In a preferred embodiment, the increased exposure of the culture medium and/or organism surface area to oxygen results in increased aeration, growth and subsequent separation of organisms from culture medium.

It is another object of the present invention to provide a centrifuge microplate fermenter with each “well” having side and bottom baffles where the centrifuge microplate has an increased shelf-life and usage life during transport, storage and sample processing.

It is yet another object of the present invention to provide a method for improving cell culturing, the method comprising:

providing a centrifuge microplate fermenter with each “well” having side and bottom baffles having capable of being sealed with breathable mats or adhesive covers.

placing cell culture media and one or more inocula cells inside the centrifuge microplate fermenter;

allowing one or more cells to divide in the media to produce progeny cells;

centrifuging the centrifuge fermenter array; and

removing the cell culture media by aspiration thereby leaving the cultured cell pellet for further use.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 illustrates a longitudinal cross section view of the centrifuge microplate fermenter.

FIG. 2 illustrates a top view of the centrifuge microplate fermenter of FIG. 1.

FIG. 3 illustrates a longitudinal cross section view of a single well of the centrifuge microplate fermenter of FIG. 1.

FIG. 4 illustrates a magnified cross section view of the encircled bottom of the centrifuge microplate fermenter of FIG. 1.

FIG. 5 illustrates a magnified top view of the of the centrifuge microplate fermenter of FIG. 1.

FIG. 6 illustrates the results of a cell growth comparison study performed using the centrifuge microplate fermenter and standard 15 ml cell culture centrifuge microplate.

### DETAILED DESCRIPTION OF THE INVENTION

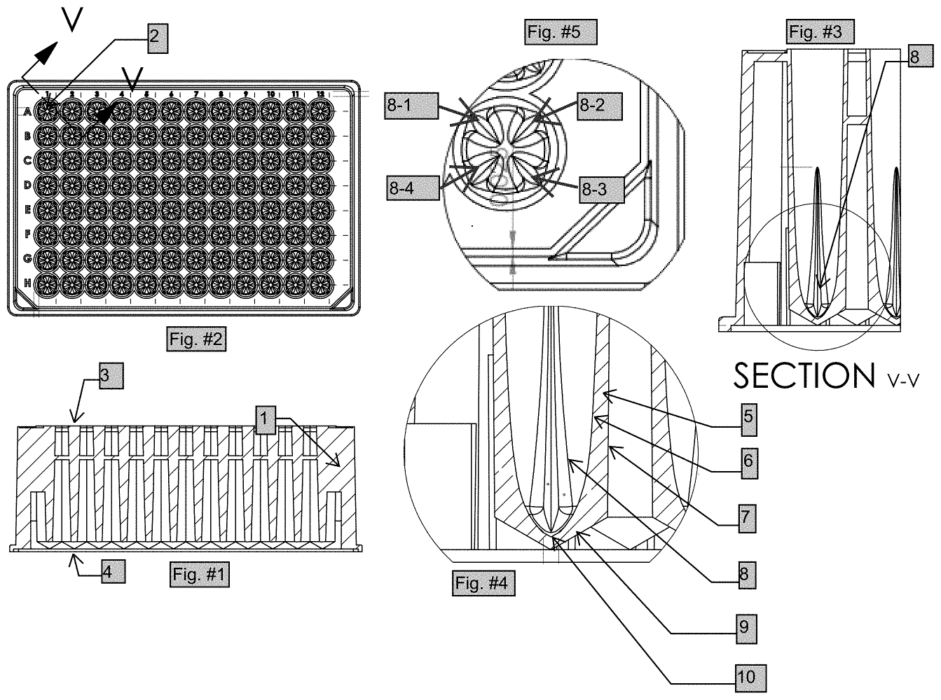

A centrifuge microplate fermenter 1 for culturing cells in a nutrient medium is shown in FIG. 1. Centrifuge microplate fermenter is comprised of a plurality of wells 2 as shown in FIG. 2. Centrifuge microplate fermenter consists of a top end 3 and a bottom end 4, the top end 3 and the bottom end 4 terminating in a conical tip 9

Centrifuge microplate fermenter is further comprised of a vertical cylindrical wall 5 positioned between top end 3 and bottom end 4. Vertical cylindrical wall 5 is further comprised of an inner wall 6 and outer wall 7. Bottom end 4 is further comprised of a plurality of baffles 8-1 which protrude from inner wall 6 toward the theoretical center of the vertical cylindrical wall 5.

With reference to FIG. 4, a magnified view of the bottom end of the centrifuge microplate fermenter 1 shown in FIG. 1 is illustrated. The conical tip 9 is visible beneath the plurality of baffles 8-1 as is the theoretical center 10 of the vertical cylindrical wall 5.

With reference to FIG. 5, a top view of the centrifuge microplate fermenter of FIG. 1 is illustrated. The position of the plurality of baffles (8-1, 8-2, 8-3, 8-4) is shown in relation to the theoretical center 10 of the vertical cylindrical wall (not shown). A plurality of baffles equals four baffles for the 2 ml centrifuge microplate fermenter as shown. The number of baffles may be increased or decreased according to the volume of the centrifuge microplate and the amount of increased aeration desired where the number of baffles will be at least one.

### Example 1

Small-scale preparation of plasmid DNA was performed in the centrifuge microplate fermenter of the present invention and a standard culture centrifuge microplate to compare cell growth/plasmid yield.

### Methods

A single bacterial colony was transferred into 0.5 ml of amp LB medium, containing 50 μg/ml ampicillin in multiple wells of 2.0 ml centrifuge microplate fermenter and standard microplate. Cell cultures were incubated overnight at 37° C. with vigorous shaking. Culture centrifuge microplates were centrifuged at 12,000 g for 30 seconds at 4° C. The supernatant was removed by aspiration and the cells in the bacterial pellet were counted and compared.

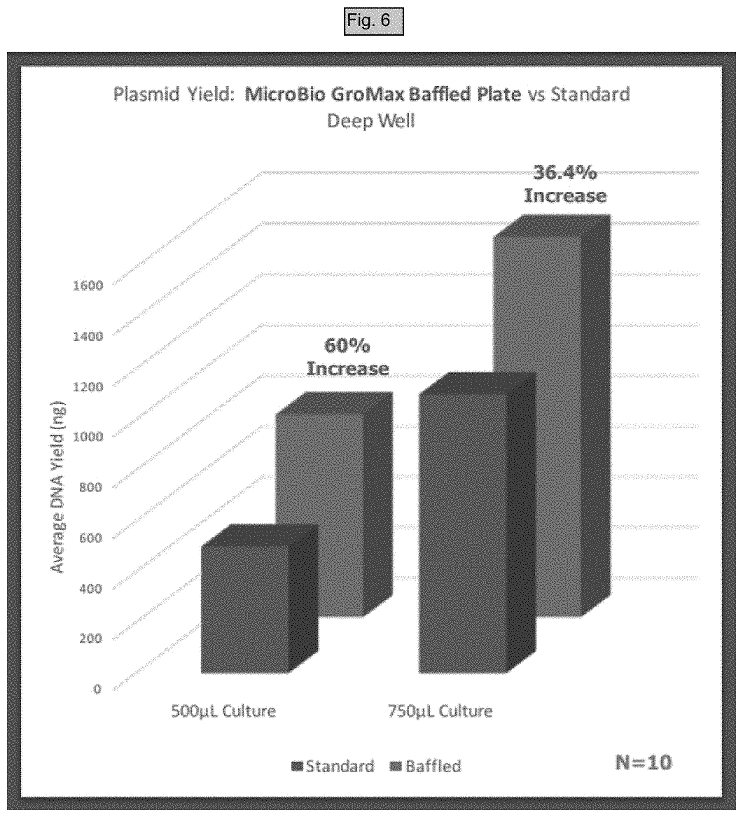

FIG. 6 shows the results of the DNA yield comparison study. DNA yield of cultures grown in standard microplate are shown in the front row while the DNA yield of cultures grown in the centrifuge microplate fermenter of the present invention are shown in the back row. The DNA yield in the centrifuge microplate fermenter increased by over 60% compared to the standard microplates. These results confirm that the design of the centrifuge microplate fermenter drastically increases bacterial culture growth resulting in a 60% increase in DNA yield per unit volume.

One embodiment of the present invention provides a centrifuge microplate fermenter for culturing cells in a nutrient medium, the centrifuge microplate fermenter comprised of:

a top end

a bottom end;

a vertical cylindrical outer wall positioned between the top end and the bottom end; and

an inner wall positioned inside the outer wall,

wherein the bottom end is further comprised of a plurality of baffles which protrude from inner wall toward a theoretical center of the vertical cylindrical outer wall

In another embodiment of the present invention, the centrifuge microplate fermenter can be fabricated from any appropriate material, including but not limited to, polypropylene/polysulfone, polyethersulfone, AB S/polycarbonate, a polyacrylic plastic and/or the like. In a preferred embodiment, the centrifuge microplate fermenter is injection molded from virgin material polyolefin, such as high-density polypropylene or polyethylene. In a most preferred embodiment, the centrifuge microplate fermenter is injection molded from ultra-clear medical grade polypropylene.

In yet another embodiment of the present invention, the centrifuge microplate fermenter may be fabricated in a variety of volumes ranging from 0.6 ml to 50 ml. In a preferred embodiment, the centrifuge microplates are centrifuge microplates with a volume ranging from 250 μl to 2 ml. In a preferred embodiment, the centrifuge microplates can be arranged and molded into 6, 12, 24, 48, 96, 192, 288, 384 well array configurations. In the most preferred red embodiment, the micro-centrifuge centrifuge microplate has a volume of 1-2 ml and can be arranged and molded in a 96 well array.

It will be appreciated that details of the foregoing embodiments, given for purposes of illustration, are not to be construed as limiting the scope of the invention. Although several embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is further defined in the converted utility application and appended claims. Further, it is recognized that many embodiments may be conceived that do not achieve all the advantages of some embodiments, particularly preferred embodiments, yet the absence of advantage shall not be construed to necessarily mean that such an embodiment is outside the scope of the present invention.

What is claimed is:

1. A centrifuge microplate fermenter with plurality of wells for culturing cells in a nutrient medium, the centrifuge microplate fermenter comprised of:
a top end
a bottom
four side walls
a plurality of wells
each well having a vertical cylindrical outer wall positioned between the top end and the bottom end; and an inner wall positioned inside the outer wall, wherein the bottom end is further comprised of a plurality of baffles which protrude from inner wall toward a theoretical center of the vertical cylindrical outer wall terminating in a conical tip.

2. The centrifuge microplate fermenter of claim 1, wherein the centrifuge microplate fermenter is fabricated from an appropriate material selected from the group consisting of polypropylene/polysulfone, polyethersulfone, ABS/polycarbonate, poluolefin, polyethylene and polyacrylic plastic.

3. The centrifuge microplate fermenter of claim 1, wherein the centrifuge microplate fermenter is fabricated by injection molding.

4. A centrifuge microplate fermenter for culturing cells in a nutrient medium, the centrifuge microplate fermenter comprised of: a top end, a bottom end; four side walls and a plurality of wells each having a vertical cylindrical outer wall positioned between the top end and the bottom end; and an inner wall positioned inside the outer wall, wherein the bottom end is further comprised of a plurality of baffles which protrude from inner wall toward a theoretical center of the vertical cylindrical outer wall terminating in a conical tip.

5. The centrifuge microplate fermenter of claim 1, wherein there are a plurality of (2-4) individual baffles that extend upward from the conical tip ½-¾ the length of the vertical cylindrical inner wall (see FIG. 4, 8-1).

6. The centrifuge microplate fermenter of claim 5, wherein each individual baffle starts in the approximate middle of the conical tip and tapers vertically and horizontally up the inner wall toward the top (open) end to a point of termination (see FIG. 4, 8-1).

7. The centrifuge microplate fermenter of claim 5, wherein the plurality of baffles take up approximately ¼-½ of the vertical cylindrical inner wall radial diameter (see FIG. 5, 8-1, 8-2, 8-3, 8-4)

8. The centrifuge microplate fermenter of claim 1, wherein the centrifuge microplate fermenter is fabricated with a total volume selected from the range of about 250 ul to about 50 ml.

9. A method for improving cell culturing, the method comprising: providing a centrifuge microplate fermenter with side and bottom baffles terminating in a conical tip; placing cell culture media and one or more inocula cells inside the centrifuge microplate fermenter; allowing one or more cells to divide in the media to produce progeny cells; centrifuging the centrifuge fermenter tube; and removing the cell culture media by aspiration thereby leaving the cultured cell pellet for further use.
